(19) United States
(12) Patent Application Publication
(10) Pub. No.: US 2004/0002772 A1
(43) Pub. Date: Jan. 1, 2004

(54) Tissue equivalents with perforations for improved integration to host tissues and methods for producing perforated tissue equivalents

(75) Inventor: Michael L. Sabolinski, Franklin, MA (US)

Correspondence Address: HALE AND DORR, LLP, 60 STATE STREET, BOSTON, MA, 02109

(73) Assignee: Organogenesis, Inc., Canton, MA (US)

(21) Appl. No.: 10/367,486

(22) Filed: Feb. 14, 2003

Related U.S. Application Data
(63) Continuation of application No. 08/431,066, filed on Apr. 28, 1995, now abandoned.

(51) Int. Cl. A61F 2/02

(52) U.S. Cl. 623/23.76

(57) ABSTRACT

This invention is directed to tissue equivalent prostheses containing perforations, which when grafted to a mammalian host, serves as a replacement body part or tissue structure while integrating with the tissue of host. The perforations provide channels for cellular ingrowth thus improving integration of the tissue equivalent to the host tissue. The prostheses of this invention, in their various embodiments, thus have dual properties: first, they function as a substitute body part, and second, they function as a template for cellular ingrowth and bioremodeling by the host cells.

### FIELD OF THE INVENTION

This invention relates to tissue equivalents made using tissue engineering technology for grafting. There are many conditions where a skin replacement can be indicated, for example: burn, congenital giant nevus, reconstructive surgery, and chronic wounds. Vascular replacements are necessary to replace damaged or diseased vessels. By providing perforations to tissue equivalents, both wound maintenance, graft take and tissue equivalent integration are enhanced. The cultured tissue is an in vitro model of the equivalent human tissue, which can be used for transplantation or implantation, in vivo, or for screening compounds in vitro.

### BACKGROUND OF THE INVENTION

Before the development of a commercially viable skin equivalent, autografts and cadaver skin allografts were used for the purposes of skin grafting. Consultation with burn surgeons indicates interest in developing an adjunct to the use of meshed autograft alone in burn patients. Currently, standard medical care calls for the use of meshed autograft alone or meshed autograft covered with meshed allograft. (Alexander, J. W., et al. (1981) J. Trauma. June; 21(6): 433-438. Kreis, R. W., et al (1989) J. Trauma. January; 29(1): 51-54. Ersek, R. A., et al (1984) Ann. Plast. Surg. December; 13(6): 482-487.)

The extent of the mesh is determined by the extent of the burn injury and the availability of donor tissue. Widely meshed autografts are used when there is very little of the patient's own tissue available for grafting and are generally covered with allograft. Sheet autografts are routinely meshed to allow for better drainage post-operatively preventing the accumulation of fluid and reducing the risk of infection. Allograft has been shown to decrease the incidence of infection, improve the take of the meshed autograft, and aid the healing process. Routine care of an unmeshed graft site may require a technique known as “pie crusting” whereby a needle or a scalpel is used to perforate a sheet graft in order to allow for drainage to occur in the event of fluid accumulation. In patients where edema is a potential problem, meshed split thickness skin grafts are the standard of care, done at the discretion of the attending physician where edema is believed to be a major concern pre-operatively.

Allograft tissue is often unavailable, costly, and unable to be rigorously screened for adventitious pathogens. Rejection of allograft within the first two weeks of application and its subsequent removal by the end of three weeks is the norm. Often surgical procedures are needed for its excision.

Widely meshed autografts have been shown to have inferior survival rates when compared to more narrowly meshed autografts (Alsbjorn, B. F., et al (1986) Ann. Plast. Surg. December; 17(6): 480-484). In some cases, there simply is not enough autologous tissue available for grafting. There exists a need to improve the survival of such grafts.

It would be advantageous to have a manufactured product which undergoes well controlled and reproducible testing for pathogens, may be made in potentially unlimited supply, and will not show clinical or immunologic signs of rejection.

In vitro technology has developed tissue equivalents for the purposes of in vitro testing or in vivo grafting for wound repair. Methods of producing such tissue equivalents are disclosed in U.S. Pat. Nos. 4,485,096, 4,485,097, 4,604,346, 4,835,102, 5,374,515 and U.S. Ser. No. 08/193,809. The present inventors have discovered a modification of cultured living skin equivalents that is applicable to a number of medical indications, one that is surprisingly effective and leads to improved wound maintenance and graft take. The improvements may cut down on the number of surgical procedures for grafting and concomitant length of hospital stays. Improved cosmetic outcome may necessitate fewer reconstructive procedures and less time spent for counseling and outpatient therapy.

Each year, approximately 300,000 coronary bypass procedures are performed in the United States. The typical treatment for small diameter artery replacement has been for surgeons to use the patient's own vessels, usually the saphenous vein from the leg. However, in many cases, the use of patient's own vessels is not practical because the veins are either damaged, diseased or are not available. In these cases, synthetic materials are used but with unsatisfactory long term results.

### SUMMARY OF THE INVENTION

The present invention overcomes the difficulties of the materials currently available and provides improved tissue equivalent grafts for replacement body tissues. This invention is directed to tissue grafts containing perforations that improve integration of the grafted tissue equivalent. The perforations provide the benefits of allowing cellular migration into the tissue equivalent. Depending on the indication, the size of the perforations provide other benefits relating to wound maintenance and graft take.

One embodiment of the invention is directed to a skin graft, which, when grafted into a patient, provides protection of the wound while integrating with patient's tissue. Indications where skin grafting is necessary are acute wounds to skin tissue due to burn injury, dermatological surgery and chronic wounds due to ulceration. The skin graft is a cultured skin equivalent, comprising cultured cells derived from skin to form a contiguous sheet. Meshing of the graft provides further benefits of conformity of the graft to the contours of the wound bed and better drainage of exudate from the wound bed. It is also possible that meshing also provides for better penetration of topical antibacterials and antifungals and increased release of cytokines and growth factors from constituent fibroblasts and keratinocytes.

Another embodiment of the invention is directed to a vascular graft, which, when grafted into a patient, functions as blood vessel, maintaining function and strength while integrating with patient's tissue. The vascular graft is constructed from bovine collagen and collagenous tissue derived from porcine small intestine. Pores are provided to the graft to allow for cellular ingrowth and remodeling of the graft.

It is, therefore, an object of this invention to provide a tissue equivalent for grafting that does not exhibit many of the shortcomings associated with many of the grafts now being used clinically.

Another object is the provision of a skin graft that will allow for improved graft take, graft persistence and integration with the patient's own tissue.

### DESCRIPTION OF THE FIGURES

FIG. 1A: Scanning electron micrograph (SEM) of a laser drilled 35 micron pore in a vascular prosthesis at 1000× magnification.

FIG. 1B: Scanning Electron micrograph of a laser drilled 35 micron pore in a vascular prosthesis at 3000× magnification.

FIG. 2: Masson's trichrome stain (cross section) of the midsection of a laser drilled vascular prosthesis (5 cm) implanted as an aortic bypass graft in a canine 30 days post-implantation at 50× magnification.

### DETAILED DESCRIPTION OF THE INVENTION

Tissue engineering is an emerging area which utilizes collagen, collagenous tissues and cultured tissue cells to construct tissue equivalents which can be used to form graftable tissue.

Living tissue equivalents have been described extensively in many patents, including U.S. Pat. Nos. 4,485,096; 4,485,097; 4,539,716; 4,546,500; 4,604,346; 4,837,379; 5,374,515 and U.S. Ser. No. 08/193,809; all of which are incorporated herein by reference.

One successful application of a living tissue equivalent is called the “Living Skin Equivalent,” which has a morphology similar to actual human skin. The Living Skin Equivalent (LSE) is composed of two layers: the upper portion is made of differentiated and stratified human epidermal keratinocytes that cover a lower layer of human dermal fibroblasts in a collagen matrix (Parenteau, et al., J. of Cellular Biochemistry, 45:245-251 (1991); Parenteau, et al., Cytotechnology, 9:163-171 (1992); and Bell et al., Toxic. in Vitro, 5:591-596 (1991)). LSE for grafting is under investigation in clinical trials for indications relating to partial and full thickness skin wounds: excision surgery, burns, venous stasis ulcers, diabetic ulcers, decubitus ulcers, and chronic inflammatory ulcers. The LSE is a full-thickness, bilayered, in vitro engineered skin tissue.

As used herein, the term “cultured tissue equivalents” means tissue equivalents of mammalian tissues, wherein the tissue equivalents are made by in vitro techniques and are meant to include monolayer skin equivalents, either a dermal equivalent or an epidermal sheet; bilayered skin equivalents, particularly LSE; and trilayered cornea equivalents and skin equivalents. The morphology of the cultured tissue equivalents are similar to the in vivo mammalian organ, typically the human organ. For illustration, the morphology of the LSE bears many similarities to human skin. Metabolically and mitotically active human dermal fibroblasts (HDF) are found throughout the dermal layer of the construct, and have been shown to secrete collagen and other matrix components into the lattice. The epidermis consists of a basal layer shown to divide with a mitotic rate similar to that of human skin. The suprabasal epidermis shows the same strata as skin in vivo, with well defined spinous and granular layers containing keratohyalin and lamellar granules covered by a stratum corneum. Immunohistochemistry demonstrates the presence of extracellular matrix components routinely found at the dermo-epidermal junction in normal human skin, such as laminin, Type IV collagen and kalanin (GB3).

Cryopreservation of living tissue equivalents has been described in U.S. Ser. Nos. 08/121,377 and 08/380,099. Cryopreservation protocols provide for long term storage and distribution of living tissue equivalents to burn centers and hospitals.

The term ‘autograft’ is an autologous tissue or organ graft; a graft that is transferred to a new position in or on the body of the same individual. Cultured autografts are skin equivalents that are prepared from a patient's own cells. The terms ‘allograft’ and ‘homograft’ can be used interchangeably and refer to a graft transplanted between genetically non-identical, or homologous, individuals of the same species. Cadavers are a typical source of allograft material, whereas cultured allografts are skin equivalents that are prepared from donor cells of another individual. The living skin equivalent of the present invention is a bilayered cultured skin equivalent.

The term ‘meshing’ is defined as a mechanical method by which a tissue is perforated with slits to form a net-like arrangement. Meshing is preferably obtained by the use of a conventional skin mesher (ZIMMER®; BIOPLASTY®). One could also manually score or perforate a tissue with a scalpel or a needle. Meshed skin may be expanded by stretching the skin so that the slits are opened and then applied to the wound bed. Expanded meshed tissue provides a wound area with maximal coverage. Alternatively, meshed skin may be applied without expansion; simply as a sheet with an arrangement of unexpanded slits. Autologous skin is in very short supply and is routinely expanded for maximal surface area coverage. Cultured living skin equivalents are potentially in unlimited supply so there is no need to expand meshed skin equivalents for the purposes of conservation. ‘Mesh ratio’ is defined as the difference of the expanded tissue as compared to its size before expansion. The length of the incisions determines the degree of expansion.

Tissue equivalents may also have perforations or fenestrations and pores provided by other means. Fenestrations may be applied manually using a punch, scalpel, needle or pin. Smaller and more uniform holes may be applied using a laser or by chemical etching.

‘Graft persistence’ is considered the continuing existence of the graft in the wound area. ‘Graft take’ is considered to be the incorporation of the graft to patient without immunological response.

Patients with burn injury or elective dermatological surgery undergo immunological testing of blood consisting of an HLA antibody screen and the presence of bovine collagen antibodies.

For burn indications, the burned wound sites to be grafted are to be prepared for the graft according to standard practice so that the burned skin area is completely excised. Excised beds will appear clean and clinically uninfected prior to grafting.

For deep partial thickness wounds due to surgical excision, the preoperative area is shaved, if necessary, cleansed with an antimicrobial/antiseptic skin cleanser (Hibiclens®) and rinsed with normal saline. Local anesthesia usually consists of intradermal administration of lidocaine/epinephrine. Once anesthesia is accomplished, a dermatome is used to remove skin to an appropriate depth, creating a deep partial thickness wound. Hemostasis can be achieved by compression with epinephrine containing lidocaine and by electrocautery.

This target site for grafting of LSE may either be directly on the excised wound bed or over meshed autograft that has been expanded at a ratio preferably between 1:1 and 3:1, more preferably at a ratio between 1:1 and 1.5:1.

The LSE should be removed from its carrier using aseptic technique, cut, if necessary, to the appropriate graft size to fit to the excised wound. The LSE is then meshed, preferably by use of a meshing apparatus. The mesh ratio of LSE should be preferably between 1:1 and 3:1, more preferably at a ratio between 1:1 and 1.5:1. The graft is applied to the wound bed and may be held in place by either staples or sutures and covered with an appropriate dressing.

Primary wound dressing should be one that is nonadherent to the LSE graft site to prevent adherence of the LSE. Secondary dressings may include absorbent dressings and/or pressure wraps, depending on the indication.

Appropriate post-operative care will be provided to the patient in examination, cleaning, changing bandages, etc. of the grafted wounds. A complete record of the condition of the grafted sites will be maintained to document all procedures, necessary medications, frequency of dressing changes and any observations made. Physical and occupational therapy is done at the investigator's determination of patient need.

Meshing the LSE prior to grafting allows for the ability greater conformability of the graft to the contours of the patient being grafted. The slits allow for the graft to remain fixed to the wound bed as they are able to open and shift with the patient's body movements without stressing or disrupting the adhered portions of the graft.

Meshed grafts allow for better drainage post-operatively by preventing the accumulation of fluid, thus reducing the risk of infection. Without preoperative meshing, the graft site may require a technique known as “pie crusting” whereby a needle or a scalpel is used to perforate a sheet graft in order to allow for drainage to occur in the event of fluid accumulation.

Clinical evidence shows that the cultured skin substitutes do provide benefit to the patient. It is postulated that cytokines and growth factors from constituent fibroblasts and keratinocytes may be likely candidates for any success in wound healing. Production of TGF-β and IL-8 have been implicated in playing key roles in wound repair. Also, better penetration of topical antibacterials and antifungals to the underlying wound is possibly obtained through the mesh. In skin graft applications, the cosmetic result is improved, thus raising the standard of care for these indications.

Many investigators believe that skin allografts cannot persist for more then 7 days and that their benefits are limited at best. Macroscopic evidence from clinical trials suggests that cultured skin substitutes might persist longer than previously thought. DNA analysis confirms prolonged persistence of the cultured skin graft. The meshing of a cultured skin equivalent provides a number of unexpected benefits.

Tissue equivalents derived from collagen and collagenous tissues are described in U.S. Pat. Nos. 5,106,949, 5,256,418 and 5,378,469 and U.S. Ser. Nos. 08/177,618, 08/215,760 and 08/417,868, all of which are incorporated herein by reference. Preclinical studies have been done for a vascular tissue equivalent, described in U.S. Ser. No. 08/177,618, for use as a blood vessel replacement. In the construction of the prosthesis, pores are provided to the construct by use of a laser. The structural layer is made of intestinal collagen layer (ICL) which is wrapped around a mandrel a number of times, preferably 2 or 3 times, to form a tubulated ICL construct. A thin layer of bovine type-I dense fibrillar collagen (DFC) is then applied to the ICL. Methods for DFC deposition are described in U.S. Pat. No. 5,256,418. The construct is then crosslinked, preferably by EDC, and the completed two or three layer prosthesis which laser drilled to create micron sized transluminal pores through the completed prosthesis for aid in cell ingrowth using an excimer laser at either KrF or XeF wavelengths. The pore size can vary from 5 to 60 microns, but is preferably less than 20 microns. Spacing of the pores can vary, but about 500 microns on center is preferred.

Sterilization of collagen and collagenous tissues is disclosed in U.S. Ser. No. 08/177,618.

The following examples are provided to better elucidate the practice of the present invention and should not be interpreted in any way to limit the scope of the present invention. Those skilled in the art will recognize that various modifications can be made to the methods described herein while not departing from the spirit and scope of the present invention.

### EXAMPLES

### Example 1

Meshing a Living Skin Equivalent

The LSE was removed from its carrier using aseptic technique and cut, if necessary, to the appropriate graft size to fit the excised wound. The ZIMMER® skin graft carrier was aseptically removed from its sterile package and placed smooth side up on the table. The LSE was placed on the carrier and spread evenly along the surface of the carrier. The hinged handle of the mesher was locked and closed prior to inserting the carrier. The carrier with the LSE was then placed on the guidance plateau of the ZIMMER® skin graft mesher with the surface of the LSE facing up. The tracks on the bottom of the carrier were aligned with the guidance plateau to ensure straight entry into the gap between the cutter and knurled roller. The leading edge of the carrier was introduced firmly between the cutter and the roller to make sure that it was straight. The ratchet handle was slowly rotated back and forth, from the 10 o'clock to 2 o'clock position, to mesh the living skin equivalent. Once meshed, the living skin equivalent was applied to the patient.

### Example 2

LSE Grafted to Burn Injuries

Patients with burn injury were entered into the burn study once he/she had fulfilled all criteria and passed all pre-graft examination for entry. 37 patients were grafted with meshed LSE over meshed autograft.

LSE was applied only to wounds that had treatment within 7 days of burn injury; and, applied only to a primary site, not to a wound bed which had previously undergone regrafting. Patients with burn wound(s) requiring grafting were selected, and target treatment sites for placing up to 2 LSE of 4 inch×8 inch size were identified. LSE were either placed directly on the excised wound bed or over meshed autograft expanded at a ratio of 2:1 or more. If the treatment site was LSE directly on the wound bed, then the control site was either autograft or allograft. If the treatment site was LSE over meshed autograft, then meshed autograft alone or covered with allograft served as the control. Test sites and control sites of meshed autograft were expanded to the same extent. LSE and allograft, when used, were of the same mesh ratio. Target treatment and control sites were randomized to left/right and top/bottom. The burned wound sites to be grafted were prepared for the graft according to standard practice so that the burned skin area was completely excised. Excised beds appeared clean and clinically uninfected.

The LSE was removed from its carrier using aseptic technique and cut, if necessary, to the appropriate graft size to fit the wound area. The LSE and allograft were then meshed separately using the method described in Example 1. The meshed LSE was placed dermal side down, unexpanded, over the autograft. The wound was appropriately dressed according to the burn units standard procedure, typically including mesh gauze, petrolatum coated gauze or the dressing.

Appropriate post-operative care was provided to the patient in examination, cleaning, changing bandages, etc. of the grafted wounds. A complete record of the condition of the grafted sites was maintained to document all procedures, necessary medications, frequency of dressing changes and any observations made. Physical and occupational therapy was done at the investigator's determination of patient need.

### Example 3

LSE Grafted to Excised Wound Injuries

A human full-thickness surgical excision wound model was used to evaluate LSE. Patients who elected voluntary dermatological surgery were enrolled once he/she had fulfilled all criteria and passed all pre-graft examination for entry. In this study, 15 patients were enrolled; 5 patients received meshed LSE and 10 received LSE that was fenestrated manually with a scalpel.

The pre-operative area was cleansed with an anti-microbial/antiseptic skin cleanser (Hibiclens®) and rinsed with normal saline. Deep partial thickness wounds were made in the skin due to surgical excision of a tattoos.

The LSE was removed from its carrier using aseptic technique and cut, if necessary, to the appropriate graft size to fit the excised wound. LSE were meshed according to Example 1. Once hemostasis was complete, LSE were either placed unexpanded, dermal side down, directly on the excised wound bed.

Silver sulfadiazane cream (SSD) 1% was applied covering the entire treatment area. Xeroform™ gauze dressing was layered above the SSD cream extending at least 1 cm beyond the wound margin. Surgical staples were then placed peripheral to the wound margin securing the Xeroform™ to the surrounding normal skin. A foam bolster cut to the shape of the surgical wound was coated with bacitracin zinc ointment. Using the surgical clips as an anchor, surgical 3-0 Onylon was tied over the bolster, securing it to the wound. The bolster was then covered with sterile 4 inch×4 inch surgical sponges and adhered with tape or a cohesive bandage. This bolster and bandage remained undisturbed for 1 week and was only removed by the investigator or designated staff at the first follow-up visit. Subjects were instructed to prevent shearing of the graft and to keep prolonged pressure off the graft.

Appropriate post-operative care was provided to the patient in examination, cleaning, changing bandages, etc. of the grafted wounds. Efficacy was determined at 1 week, 2 weeks, 1 month, 3 months and 12 months post-graft using acetate tracings, serial photography and assessment of graft persistence. A complete record of the condition of the grafted sites was maintained to document all procedures, necessary medications, frequency of dressing changes and any observations made.

Preliminary data showed that LSE behaved like a skin graft on patients with deep dermal wounds by gradually becoming integrated into the patient's own skin tissue.

### Example 4

Persistence of Grafted LSE Demonstrated by Polymerase Chain Reaction

The use of SSP-PCR HLA (Sequence Specific Primer-Polymerase Chain Reaction Human Lymphocyte Antigen) analysis to detect donor cells in a graft biopsy has been applied to allografts of cultured skin substitutes to address persistence. A method using sequence specific primers for A33 HLA Class I gene, was used to determine whether or not allograft survival was longer than previously thought.

Human patients in the study were grafted with meshed LSE. At time points 6 days and 13 days after grafting, a biopsy was taken. Cadaver skin was obtained from the New York Firefighters Skin Bank (New York, N.Y.). Serological typing was performed by Brigham and Women's Hospital tissue typing facility.

DNA was extracted from the punch biopsy and processed. PCR was carried out using primers that were previously developed for HLA subtypes A33 and B53/35. The sequences for the A33 specific primers were GAGTATTGGGACCGGAAC and GCGCAGGTCCTCGTTCAA. The PCR was carried out in 50 μl volumes containing: 17 mM ammonium sulfate; 67 mM Tris pH 8.0; 6.7 mM EDTA; 0.017% bovine serum albumin; 200 μM dNTP's; 2 mM MgCl₂; 0.2 μM of each primer and 0.1 μg of target DNA. The reaction mix was denatured at 95° C. for 5 min. and 1 unit of Amplitaq (Perkin Elmer Cetus) was added. The amplifications were carried out in a Perkin Elmer ThermoCylcer 480 with thin wall tubes. The cycle used was: 1 min. at 95° C.; 1 min. at 55° C.; and 1 min. at 72° C. for 30 cycles. PCR products were separated on a 2% agarose gel containing 0.5 μg of ethidium bromide. Gels were run for 30 min. at 120 V in 1×TAE buffer (40 mM Tris base, 20 mM glacial acetic acid, 2 mM EDTA pH 8.0) and the DNA was visualized by UV illumination and photographed.

The sequences for B53/35 specific primers were GGAGTATTGGGACCGGAAC and GCCATACATCCTCTGGATGA. The PCR was carried out in 50 μl volumes containing: 100 mM Tris pH 8.3; 500 mM KCl; 200 μM dNTP's; 1 mM MgCl₂; 1 μM of each primer and 0.1 μg of target DNA. The reaction mix was denatured at 95° C. for 5 min. and then 1 unit of Amplitaq (Perkin Elmer Cetus) was added. The amplifications were carried out in a Perkin Elmer ThermoCylcer 480 with thin wall tubes. The cycling was carried out as follows: 1 min. at 95° C.; 1 min. at 60° C.; and 1 min. at 72° C. for 30 cycles. PCR products were separated on a 2% agarose gel containing 0.5 μg of ethidium bromide. Gels were run for 30 min. at 120 V in 1×TAE buffer (40 mM Tris base, 20 mM glacial acetic acid, 2 mM EDTA pH 8.0) and the DNA was visualized by UV illumination and photographed.

The resulting bands were sequenced (Sequetech, Calif.) directly from PCR and comparison were made between LSE vs. patient day 13 product to confirm that we were indeed amplifying HLA-A33 sequences The specificity of the primers for HLA-A33 was tested against a panel of serologically typed individual or volunteers, LSE and a sample of cadaver skin. Patient 2 and LSE DNA was found to be A33 positive while patient 1, 3 and cadaver skin were negative. Individual 2 type had previously been shown to be HLA-A33 positive by serological typing techniques. This confirmed the primers were specific.

After establishing a source of cadaver skin DNA that was HLA-A33 negative by PCR analysis, different quantities of cadaver skin DNA and LSE DNA were mixed. As little as 1 μg of LSE DNA in 99 ng of cadaver skin DNA, could be detected by this method. This indicates that as long as 1% of the biopsy was LSE it could be detected. The HLA B53/35 were shown to detect LSE at greater than 25% of the biopsy.

The PCR of the patient biopsies were performed. DNA was isolated from each patient's peripheral blood leukocytes (PBL) as a control. DNA from LSE, patient PBL's day 7 and day 13 biopsies were analyzed by SSP-PCR-HLA using HLA-A33 primers. The patient's peripheral blood leukocytes were negative while the biopsies of the grafted area proved positive at both time points. PCR was performed again and the resulting bands from LSE and the day 13 biopsy were sequenced. The sequences match exactly.

PCR was also performed, in a like manner as given in the above paragraph, using primers specific for HLA B53/35. This primer set can not distinguish between B53 and B35, however the patient was negative for both these HLA types. The results matched the A33 results.

Persistence of allografts remains has been found to persist out to 13 days with evidence suggesting persistence out to 21 days.

### Example 5

Production of a Laser Drilled Vascular Graft Prosthesis

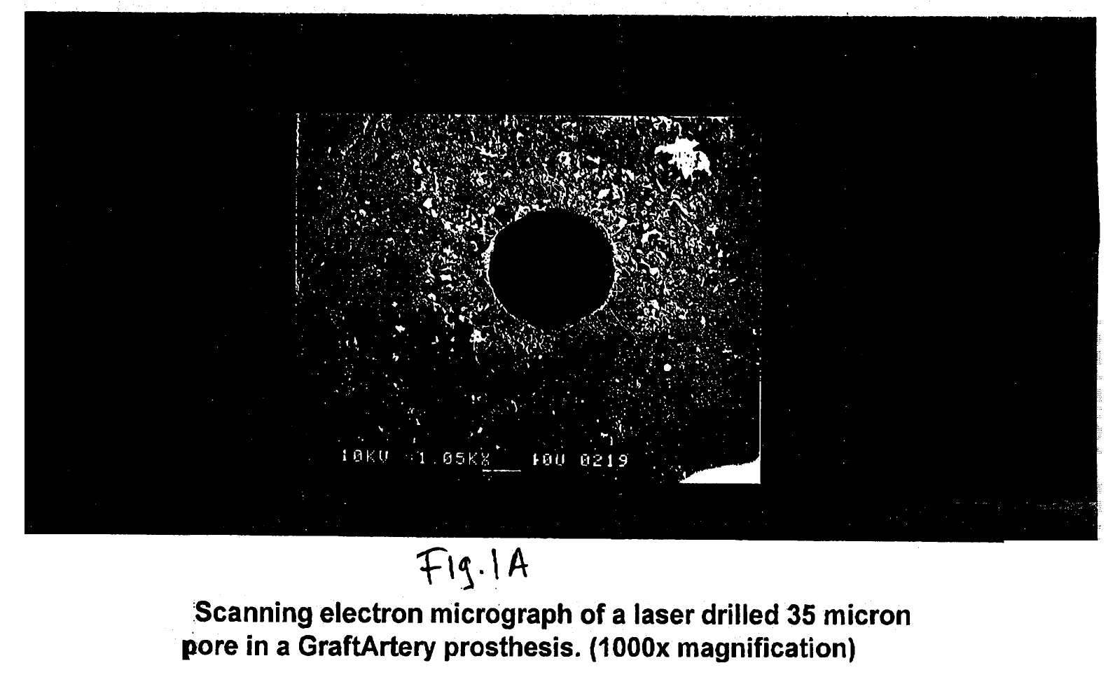

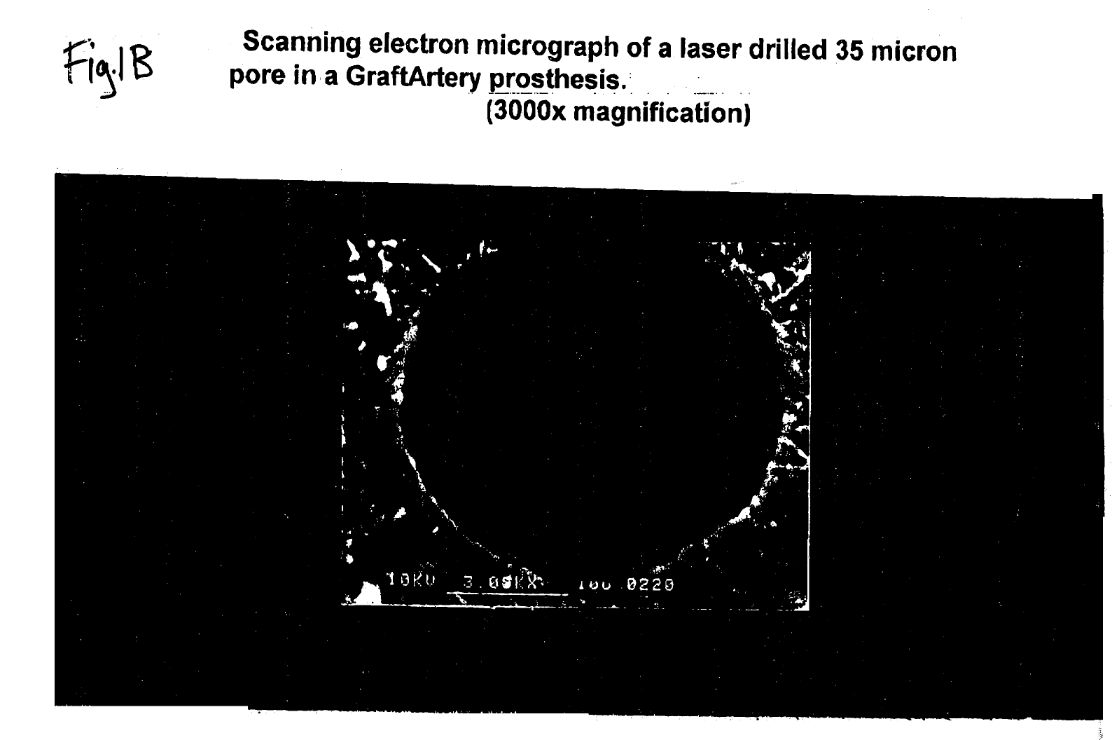

Porcine intestine was harvested, mechanically stripped, and decontaminated, leaving a strong, predominantly type-I collagenous ICL layer. The ICL was wrapped around a mandrel of the appropriate diameter, heat welded and lightly crosslinked with a water soluble carbodiimide (EDC). A thin layer bovine type-I dense fibrillar collagen (DFC) coated with benzalkonium chloride heparin was then applied to provide a smooth flow surface. Laser drilled 35 micron transluminal holes were made through the prepared grafts using a krypton fluoride (KrF) laser as shown in FIGS. 1A and 1B. The hole pattern was 500 microns on center. The grafts were peracetic acid sterilized as described in U.S. Ser. No. 08/177,618 and packaged for use.

### Example 6

The Effect of Laser Drilled Holes on the Remodeling of a Laser Drilled Vascular Graft Prosthesis

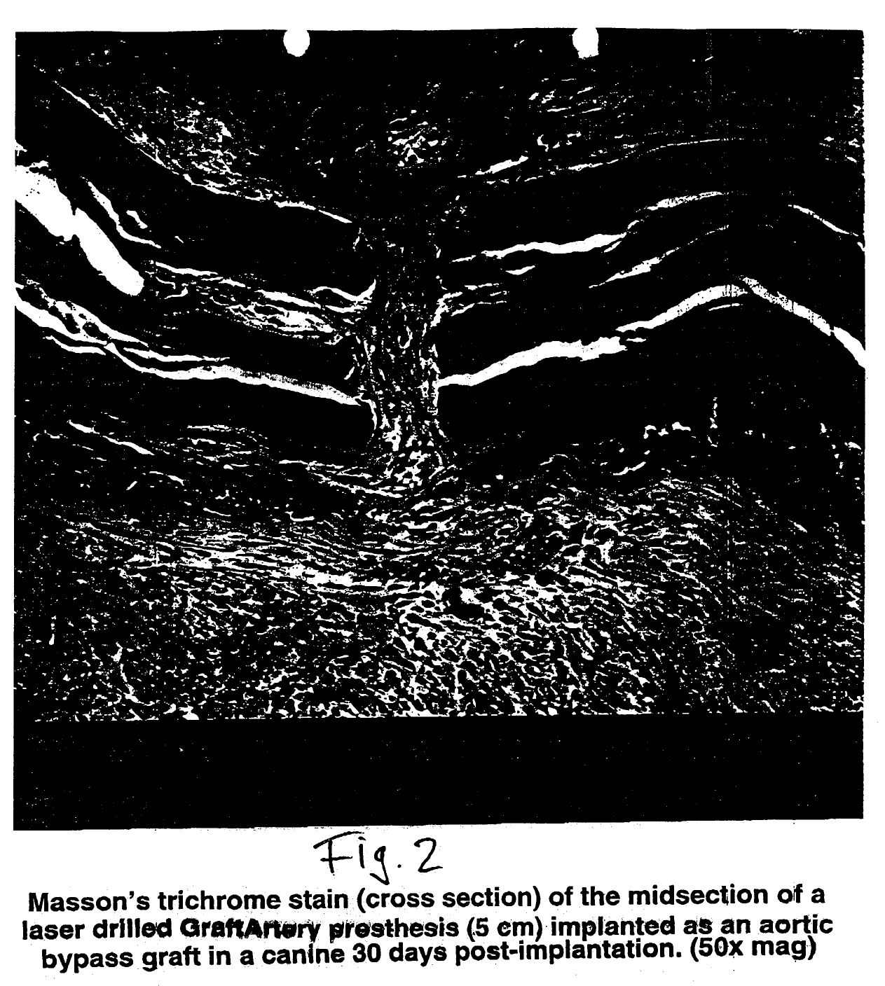

Eight laser-drilled prostheses (6 mm diameter×8 cm long), as described in Example 5, were implanted as an interposition graft in canine aorta. Grafts were harvested at 30 days, 60 days, and 90 days post implantation. Sections of each graft were processed histologically to determine the amount and type of cellular ingrowth and remodeling. At 30 days, vascular ingrowth to the lumen was seen throughout the laser drilled grafts, endothelial cells were seen spreading out from those capillaries covering the midsection of the graft, as shown in FIG. 2. By 60 days, the graft was seen to be completely covered with a confluent endothelial layer. In addition to the transmural migration of endothelial cells, the ICL and DFC collagens were seen to be remodeling faster throughout the graft due to the increased presence of smooth muscle cells within the wall of the graft.

### Example 7

Meniscus Prostheses made from ICL

A meniscus replacement was designed for a preclinical study using sheep. ICL was mechanically processed and sterilized by the peracetic acid process described in U.S. Ser. No. 08/177,618. ICL layers were prepared by trimming lymph tags, splitting the tube down the tag side and removing excess moisture. The inner segment was prepared by layering 3 pieces of ICL approximately 4 inches then rolling tightly about the long axis to form a solid cylinder. The cylinder was trimmed to about 2 inches. The outer segment was prepared by rolling a single 6 inch length over the cylinder for at least two revolutions, the excess on each end was twisted tight. The sample was curved to fit a specially designed stainless steel meniscus template such that the exposed seam was external to the curve. The sample was clamped between two meniscus templates and heat welded, as described in U.S. Ser. No. 08/417,868, at 62° C. for 55±5 minutes. The sample was quenched in a 4° C. water bath for 10 minutes prior to crosslinking by submersion in 100 mM ethyl-3-(3-dimethylaminopropyl)carbodiimide hydrochloride (EDC) overnight. The outer edge of the curved portion was punctured with an 18 gauge sharp tip needle to a maximum depth without penetrating the inner wall of the curve. Punctures were placed at 4 mm intervals along the entire length of the outer curve. The prostheses were then sterilized by cold peracetic acid process.

### Example 8

Meniscus Prostheses made from ICL and DFC

A meniscus replacement was designed for a preclinical study using sheep. A 3-bar woven fabric (0-1/1-0,1-0/4-5,4-5/1-0) of 3-ply 150 denier collagen yarn was prepared as described in U.S. Pat. No. 5,378,469 and U.S. Ser. No. 08/215,760. The fabric underwent consecutive washes in acetone and water was then air dried flat under weight. Lengths of 4.5 cm by 4 cm were wet and rolled tightly about the long axis. The sample was secured with split tubing and dried under a cool air flow. An outer sheath of ICL was added by wrapping a 6 inch length of ICL over the fabric roll for two revolutions, allowing the extra ICL on the ends wrap about itself. The sample was curved and dried such that the exposed seam was on the outside of the curve. The sample was heat welded, as described in U.S. Ser. No. 08/417,868, at 62° C. for 2 hours, then quenched in a 4° C. water bath for 10 minutes. The sample was then crosslinked by submersion in 100 mM 1-ethyl-3-(3-dimethylaminopropyl)carbodiimide hydrochloride overnight. The outer side of the curve was punctured with an 18 gauge sharp tip needle to a maximum depth without penetrating the inner wall of the curve. Punctures were placed at 4 mm intervals along the entire length of the outer curve. The prostheses were then sterilized by cold peracetic acid process.

In the description and examples given above, the referenced U.S. Patents and pending patent applications are all incorporated herein by reference. Further, although the foregoing invention has been described in some detail by way of illustration and example for purposes of clarity and understanding, it will be obvious to one of skill in the art that certain changes and modifications may be practiced within the scope of the appended claims.

What we claim is:

1. A cultured tissue equivalent for grafting with improved integration to host tissue comprising a cultured tissue equivalent with perforations provided to the tissue equivalent, wherein said cultured tissue equivalent is ready for grafting to a patient.

2. The cultured tissue equivalent of claim 1 wherein said perforations are provided by meshing.

3. The cultured tissue equivalent of claim 1 wherein said perforations are provided by applying a punch, scalpel, needle or pin to the cultured tissue equivalent.

4. The cultured tissue equivalent of claim 1 wherein said perforations are provided by applying a laser.

5. The cultured tissue equivalent of claim 1, wherein said cultured tissue equivalent comprises collagen or collagenous tissue.

6. The cultured tissue equivalent of claim 1, wherein the cultured tissue equivalent is selected from the group consisting of dermal equivalents, epidermal sheets, bilayered skin equivalents, trilayered cornea equivalents and trilayered skin equivalents.

7. The cultured tissue equivalent of claim 1, wherein the cultured tissue equivalent is living skin equivalent comprising keratinocytes and fibroblasts.
